(19) United States
(12) Patent Application Publication
(10) Pub. No.: US 2006/0089645 A1
(43) Pub. Date: Apr. 27, 2006

(54) Internal fixation system for spine surgery

(75) Inventor: Walter W. Eckman, Tupelo, MS (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Concept Matrix, LLC

(21) Appl. No.: 11/258,778

(22) Filed: Oct. 26, 2005

Related U.S. Application Data
(60) Provisional application No. 60/622,174, filed on Oct. 26, 2004.

(51) Int. Cl. A61F 2/30

(52) U.S. Cl. 606/61

(57) ABSTRACT

An internal fixation rod for spine surgery includes an elongate body, a first rod end, a second rod end, a first mounting member and a second mounting member. The first mounting member is disposed proximate the first rod end, and the second mounting member is disposed proximate the second rod end. The first mounting member and the second mounting member are smaller in cross-section than the elongate body.

### CROSS REFERENCE TO RELATED APPLICATIONS

This application claims priority to U.S. Provisional Patent Application No. 60/622,174 filed on Oct. 26, 2004, entitled “Internal Fixation System.”

### BACKGROUND OF THE INVENTION

The present invention relates to an apparatus and method for performing spine surgery and more particularly to an internal fixation system for minimally invasive and open spine surgery and a method for using the internal fixation system for spine surgery.

Referring to prior art FIGS. 1A and 1B, the spine 120, also known as the vertebral column or the spinal column, is a flexible column of vertebrae 100 (special types of bones) held together by muscles, ligaments and tendons. The spine 120 extends from the cranium (not shown) to the coccyx 126, encasing a spinal cord 128 and forming the supporting axis of the body (not shown). The spinal cord 128 is a thick bundle of nerve tissue (nerves) that branch off to various areas of the body for the purposes of motor control, sensation, and the like. The spine 120 includes seven cervical vertebrae (not shown), twelve thoracic vertebrae (not shown), five lumbar vertebrae, L¹-L^{V}, five sacral vertebrae, S¹-S^{V}, and three coccyx vertebrae 126. The sacral and coccyx vertebrae are each fused, thereby functioning as a single unit. FIG. 1B shows the lumbar region 122, the sacral region 124 and the coccyx 126 of the spine 120 and that the vertebrae 100 are stacked one upon another. The top portion 100a and bottom portion 100b of each vertebrae 100 is slightly concave. The opposing concave vertebral surfaces form the intervertebral space 121 in which an intervertebral disk (not shown) resides. Each of the intervertebral disks has a soft core referred to as a nucleus pulposus or nucleus (not shown).

In FIG. 1A, directional arrow 101a is pointing in the posterior direction and directional arrow 101b is pointing in the anterior direction. FIG. 1A shows that each vertebrae 100 includes a body 106 in the innermost portion, a spinal canal 108 and a spinous process 102 at the posterior-most end of the vertebra 100. The vertebrae 100 are substantially similar in composition, but vary in size from the larger lumbar to the smallest coccyx vertebrae 126. Each vertebrae 100 further includes two transverse processes 104 located on either side and a protective plate-like structure referred to as a lamina 110. Nerves from the spinal cord 128 pass through the spinal canal 108 and foramina 111 to reach their respective destinations within the body.

After spine surgery, adjacent vertebrae 100 may require a fixation system to be clamped to the side where the surgeon accessed the vertebrae 100. The typical fixation system includes installing pedicle screws in each vertebra 100 and securing a rigid plate or rod to the screws. The presently available systems are difficult to install through very small portals or working channels, e.g., a working channel less than one inch in diameter.

It is desirable to provide an internal fixation system for minimally invasive spine surgery and a method for using the internal fixation system. It is desirable to provide an internal fixation system for securing adjacent vertebrae that includes a fixation rod having mounting members at each end. It is desirable to provide an internal fixation system for securing adjacent vertebrae that includes a fixation rod which deviates medially and dorsally.

### BRIEF SUMMARY OF THE INVENTION

Briefly stated, the present invention comprises an internal fixation rod for spine surgery that includes an elongate body, a first rod end, a second rod end, a first mounting member and a second mounting member. The first mounting member is disposed proximate the first rod end, and the second mounting member is disposed proximate the second rod end. The first mounting member and the second mounting member are smaller in cross-section than the elongate body.

The present invention further comprises an internal fixation system for spinal surgery that includes two pedicle screws, two locking nuts and a fixation rod. Each of the pedicle screws has a bone-mating thread at a first end and a mating thread at a second end. Each of the locking nuts has a mating thread configured to mate with the mating thread of each of the pedicle screws. The fixation rod includes an elongate body, a first rod end, a second rod end, a first mounting member and a second mounting member. The first mounting member is disposed proximate the first rod end, and the second mounting member is disposed proximate the second rod end. The first mounting member and the second mounting member are smaller in cross-section than the elongate body. The first mounting member is mounted over one of the pedicle screws and secured by one of the locking nuts and the second mounting member is mounted over the other one of the pedicle screws and secured by the other one of the locking nuts.

The present invention further comprises a method of securing adjacent vertebrae. The method includes accessing a first vertebra and a second vertebra of a spine. An internal fixation rod is mounted to the first vertebra and the second vertebra. The internal fixation rod includes an elongate body, a first rod end, a second rod end, a first mounting member and a second mounting member. The first mounting member is disposed proximate the first rod end, and the second mounting member is disposed proximate the second rod end. The first mounting member and the second mounting member are smaller in cross-section than the elongate body.

The present invention also comprises a method of installing an internal fixation system for securing adjacent vertebrae. The method includes making an incision between about 10 millimeters (mm) and about 100 mm in span in a posterior region of a patient proximate a first vertebra and a second vertebra of a spine of the patient. A distal end of a working channel is inserted adjacent the first vertebra and the second vertebra of the spine accessible through the incision. The first vertebra and the second vertebra of the spine are accessed through the working channel. The internal fixation system is mounted to the first vertebra and the second vertebra. The internal fixation system includes a fixation rod. The fixation rod includes an elongate body, a first rod end, a second rod end, a first mounting member and a second mounting member. The first mounting member is disposed proximate the first rod end, and the second mounting member is disposed proximate the second rod end. The first mounting member and the second mounting member are smaller in cross-section than the elongate body.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A is a top sectional view of a human vertebrae as is known in the art;

FIG. 1B is a side sectional view of the lumbar and sacral regions of a human spine as in known in the art;

FIG. 2 is a perspective view of an internal fixation rod for minimally invasive and open spine surgery in accordance with a first preferred embodiment of the present invention;

FIG. 3 is a perspective view of an internal fixation rod for minimally invasive and open spine surgery in accordance with a second preferred embodiment of the present invention;

FIG. 4 is a front elevational view of a first internal fixation clamp in accordance with the preferred embodiments;

FIG. 5 is a rear elevational view of the first internal fixation clamp of FIG. 4;

FIG. 6 a top view of the first internal fixation clamp of FIG. 4;

FIG. 7 is a partial perspective view of the fixation rod of FIG. 2 mounted on a mating portion of a pedicle screw with the fixation clamp of FIG. 4 mounted on top thereof;

FIG. 8 is a perspective view of an internal fixation rod for minimally invasive and open spine surgery in accordance with a third preferred embodiment of the present invention;

FIG. 9 is a perspective view of an internal fixation rod mounted to adjacent vertebrae of a spine using pedicle screws, fixation clamps and locking nuts, together forming an internal fixation system in accordance with the various preferred embodiments the present invention;

FIG. 10 is a perspective view of a plurality of internal fixation rods mounted to a plurality of adjacent vertebrae of a spine using pedicle screws, fixation clamps and locking nuts, together forming an internal fixation system in accordance with the various preferred embodiments the present invention;

FIG. 11A is a side elevational view of a locking nut for use with the preferred embodiments of the present invention;

FIG. 11B is a bottom plan view of the locking nut of FIG. 1I A;

FIG. 12A is a side elevational view of a first pedicle screw for use with the preferred embodiments of the present invention;

FIG. 12B is a top plan view of the first pedicle screw of FIG. 12A;

FIG. 13A is a side elevational view of a second pedicle screw for use with the preferred embodiments of the present invention;

FIG. 13B is a top plan view of the second pedicle screw of FIG. 13A;

FIG. 14A is a side elevational view of a third pedicle screw for use with the preferred embodiments of the present invention;

FIG. 14B is a top plan view of the third pedicle screw of FIG. 14A;

FIG. 15A is a side elevational view of a fourth pedicle screw for use with the preferred embodiments of the present invention;

FIG. 15B is a top plan view of the fourth pedicle screw of FIG. 15A;

FIG. 16A is a side elevational view of a fifth pedicle screw for use with the preferred embodiments of the present invention;

FIG. 16B is a top plan view of the fifth pedicle screw of FIG. 16A;

FIG. 17A is a side elevational view of a sixth pedicle screw for use with the preferred embodiments of the present invention;

FIG. 17B is a top plan view of the sixth pedicle screw of FIG. 17A;

FIG. 18 is a side elevational view of a second fixation clamp mounted on a pedicle screw in accordance with the preferred embodiments of the present invention;

FIG. 19 is a top perspective view of the fixation clamp of FIG. 18; and

FIG. 20 is a perspective view of a working channel.

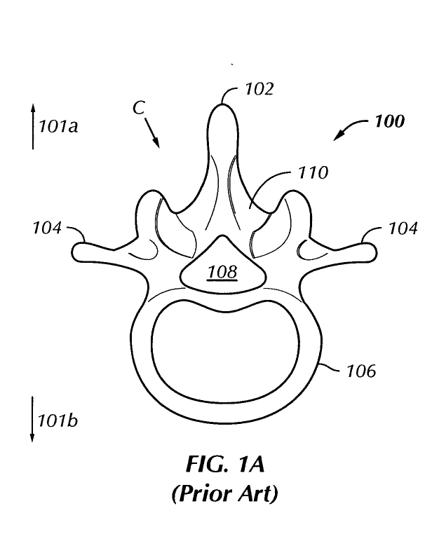

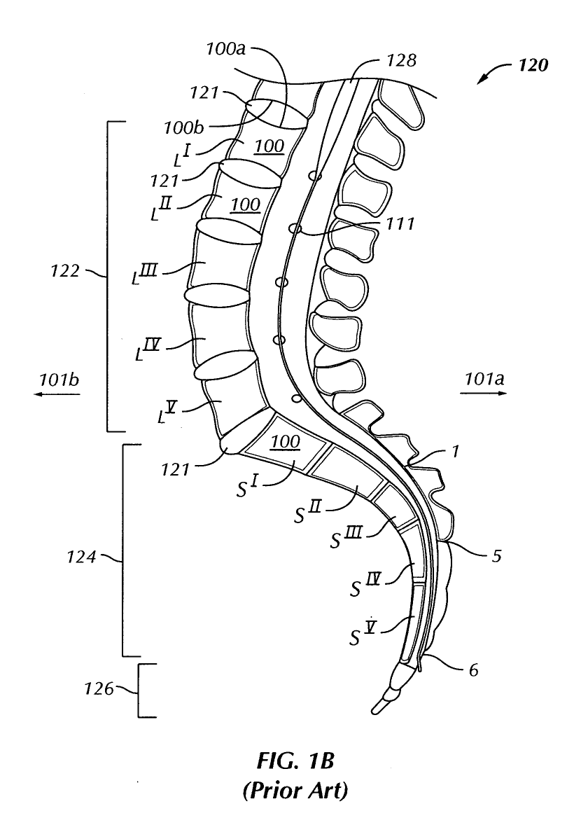

### DETAILED DESCRIPTION OF THE INVENTION

Certain terminology is used in the following description for convenience only and is not limiting. The words “right”, “left”, “lower”, and “upper” designate directions in the drawing to which reference is made. The words “inwardly” and “outwardly” refer direction toward and away from, respectively, the geometric center of the object described and designated parts thereof. The terminology includes the words above specifically mentioned, derivatives thereof and words of similar import. Additionally, the word “a”, as used in the claims and in the corresponding portions of the specification, means “at least one.”

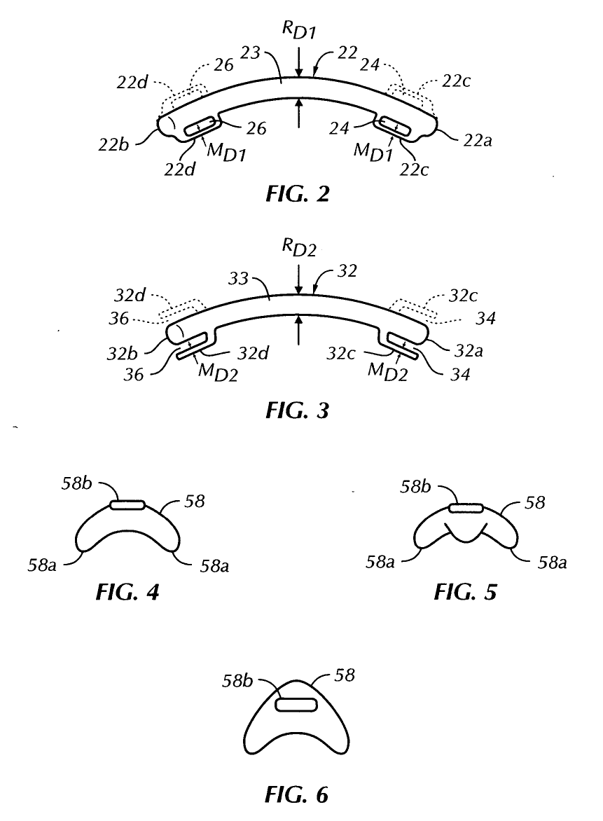

Referring to the drawings in detail, wherein like reference numerals indicate like elements throughout, FIG. 2 shows an internal fixation rod 22 for minimally invasive and open spine surgery in accordance with a first preferred embodiment of the present invention. The fixation rod 22 is an internally-mounted device, with respect to a patient, for fixing two or more adjacent vertebrae 100 after a surgical procedure such as installing a fusion cage (not shown) or the like.

The internal fixation rod 22 has an elongate body 23, a first rod end 22a, a second rod end 22b, a first mounting member 22c disposed proximate the first rod end 22a and a second mounting member 22d disposed proximate the second rod end 22b. The fixation rod 22 is about 2-5 centimeters (cm) long, but the fixation rod 22 may vary in length depending on the size and shape of the patient. The elongate body 23 of the fixation rod 22 has a diameter or cross-sectional dimension R_{D1} of about 4-7 mm, but need not have a circular cross-section. The mounting members 22c, 22d each have a diameter or cross-sectional dimension M_{D1} of about 0.1-2 mm, but need not have a circular cross-section. Thus, the first and second mounting members 22c, 22d each have a cross-sectional dimension M_{D1} that is smaller than the cross-sectional dimension R_{D1} of the elongate body 23 of the fixation rod 22. Preferably, the elongate body 23 and the first and second mounting members 22c, 22d are rounded or chamfered. The fixation rod 22 is preferably configured for minimally invasive spine surgery. But, the fixation rod 22 may also be used in conventional open surgery.

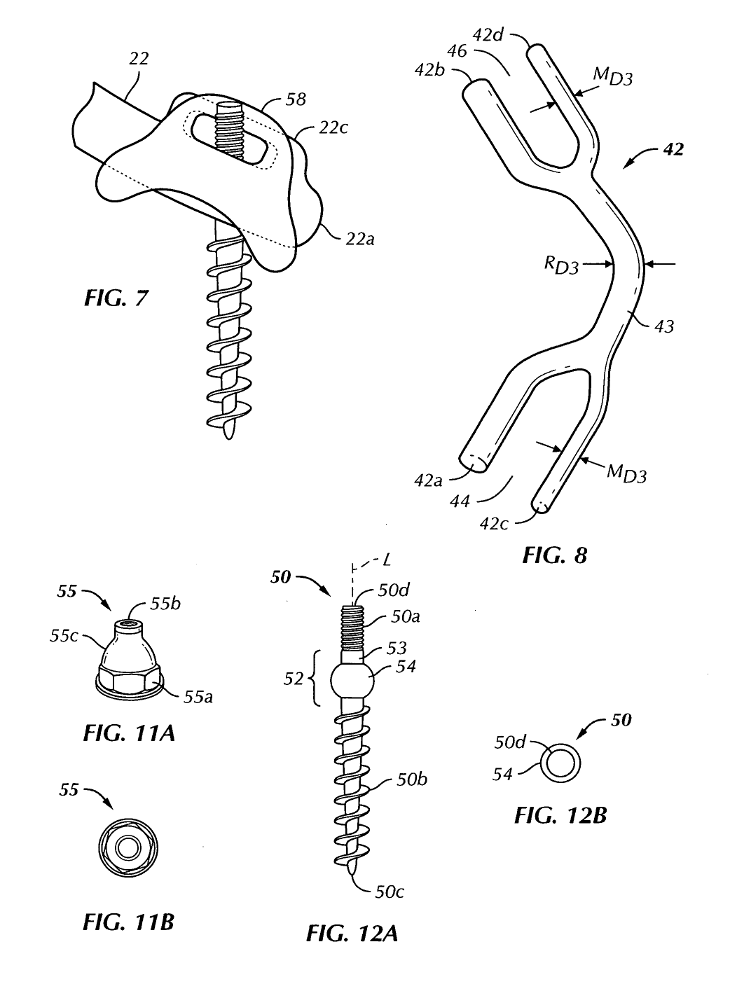

Each rod end 22a, 22b is configured to be mounted to an exposed portion of a pedicle screw 50 as shown in FIG. 7. The first and second mounting members 22c, 22d are preferably smaller in cross-sectional dimension than the main elongate body 23 of the fixation rod 22 permitting the fixation rod 22 to tilt freely at nearly any angle during installation. The first and second mounting members 22c, 22d preferably connect to the elongate body 23 of the fixation rod 22 at two places in order to form a loop or eyelet 24, 26, respectively. The first and second mounting members 22c, 22d may be mounted on either a concave side of the elongate body 23 of the curved rod 22 (shown in solid in FIG. 2) or convex side of the curved rod 22 (shown in phantom in FIG. 2). Alternately, the fixation rod 22 is generally straight and the first and second mounting members 22c, 22d may be mounted on any side of elongate body 23 of the internal fixation rod 22.

The first and second mounting members 22c, 22d are generally only structurally relied upon during installation of the internal fixation rod 22 to hold the respective first and second rod ends 22a, 22b to a pedicle screw 50 (FIGS. 12A-12B) before a surgeon fixes the respective first and second rod ends 22a, 22b.

Alternately, the first and second mounting members 22c, 22d can be a thin wire or cable because they are only structurally relied upon during installation of the internal fixation rod 22 to hold the respective first and second rod ends 22a, 22b to a pedicle screw 50 before a surgeon fixes the respective first and second rod ends 22a, 22b. The surgeon sets the internal fixation rod 22 on a fixed (nonmoving) screw 50, and the internal fixation rod 22 takes a particular tilt to accommodate the particular installation, then the surgeon fixes the respective first and second rod ends 22a, 22b.

Preferably, the internal fixation rod 22 deviates medially and dorsally between the first rod end 22a and the second rod end 22b. The fixation rod 22 deviates medially and dorsally to ease installation or adjustment of material or devices through the foramen 104 between adjacent vertebrae 100. Preferably, the deviation of the fixation rod 22 is generally arcuate, and a dorsal side of the fixation rod 22 is generally convex.

FIG. 3 shows an internal fixation rod 32 for minimally invasive and open spine surgery in accordance with a second preferred embodiment of the present invention. The fixation rod 32 is substantially similar to the fixation rod 22 of the first preferred embodiment. The fixation rod 32 has an elongate body 33, a first rod end 32a, a second rod end 32b, a first mounting member 32c disposed proximate the first rod end 32a and a second mounting member 32d disposed proximate the second rod end 32b. The first and second mounting members 32c, 32d are open-ended (i.e., only connect to the fixation rod 32 at a single point each), thereby forming a generally Y-shape or U-shape defining openings 34, 36, respectively, at each of the rod ends 32a, 32b.

The fixation rod 32 is about 2-5 cm long, but the fixation rod 32 may vary in length depending on the size and shape of the patient. The elongate body 33 of the fixation rod 32 has a diameter or cross-sectional dimension R_{D2} of about 4-7 mm, but need not have a circular cross-section. The mounting members 32c, 32d each have a diameter or cross-sectional dimension M_{D2} of about 0.1-2 mm, but need not have a circular cross-section. Thus, the first and second mounting members 32c, 32d each have a cross-sectional dimension M_{D2} that is smaller than the cross-sectional dimension R_{D2} of the elongate body 33 of the fixation rod 32. Preferably, the elongate body 33 and the first and second mounting members 32c, 32d are rounded or chamfered. Each end 32a, 32b is mounted to an exposed portion of a pedicle screw 50, similar to the fixation rod 22 that is shown in FIG. 7. The first and second mounting members 32c, 32d are preferably smaller in cross-sectional dimension than the main body of the fixation rod 32 permitting the fixation rod 32 to tilt freely at any nearly angle during installation. The first and second mounting members 32c, 32d may be mounted on either a concave side of the curved rod 32 (shown in solid in FIG. 3) or a convex side of the curved rod 32 (shown in phantom in FIG. 3). Alternately, the fixation rod 32 is generally straight and the first and second mounting members 32c, 32d may be mounted on any side of the elongate body 33 of the internal fixation rod 32.

The first and second mounting members 32c, 32d are generally only structurally relied upon during installation of the internal fixation rod 32 to hold the respective first and second rod ends 32a, 32b to a pedicle screw 50 (FIGS. 12A-12B) before a surgeon fixes the respective first and second rod ends 32a, 32b.

Preferably, the fixation rod 32 deviates medially and dorsally between the first rod end 32a and the second rod end 32b. The fixation rod 32 deviates medially and dorsally to ease installation or adjustment of material or devices through the foramen 104 between adjacent vertebrae 100. Preferably, the deviation of the fixation rod 32 is generally arcuate, and a dorsal side of the fixation rod 32 is generally convex.

FIG. 8 shows an internal fixation rod 42 for minimally invasive and open spine surgery in accordance with a third preferred embodiment of the present invention. The fixation rod 42 is substantially similar to the fixation rod 32 of the second preferred embodiment. The fixation rod 42 has an elongate body 43, a first rod end 42a, a second rod end 42b, a first mounting member 42c disposed proximate the first rod end 42a and a second mounting member 42d disposed proximate the second rod end 42b. The first and second mounting members 42c, 42d are open-ended (i.e., only connect to the fixation rod 42 at a single point), thereby forming a generally Y-shape or U-shape at each end 42a, 42b defining openings 44, 46, respectively, at each of the rod ends 42a, 42b.

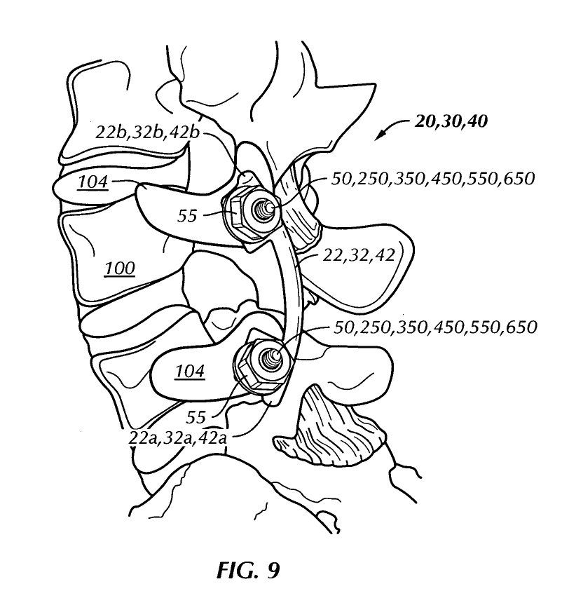

The fixation rod 42 is about 2-5 cm long, but the fixation rod 42 may vary in length depending on the size and shape of the patient. The elongate body 43 of the fixation rod 42 has a diameter or cross-sectional dimension R_{D3} of about 4-7 mm, but need not have a circular cross-section. The mounting members 42c, 42d each have a diameter or cross-sectional dimension M_{D3} of about 0.1-2 mm, but need not have a circular cross-section. Thus, the first and second mounting members 42c, 42d each have a cross-sectional dimension M_{D3} that is smaller than the cross-sectional dimension R_{D3} of the elongate body 43 of the fixation rod 42. Preferably, the elongate body 43 and the first and second mounting members 42c, 42d are rounded or chamfered. Each end 42a, 42b is mounted to an exposed portion of a pedicle screw 50 (FIG. 9). The first and second mounting members 42c, 42d are preferably smaller in cross-sectional dimension M_{D3} than the main body of the fixation rod 42 permitting the fixation rod 42 to tilt freely at nearly any angle during installation. The first and second mounting members 42c, 42d may be mounted on either a concave side of the curved rod 42 (not shown) or a convex side of the curved rod 42 (shown in solid in FIG. 8). Alternately, the fixation rod 42 is generally straight and the first and second mounting members 42c, 42d may be mounted on any side of the internal fixation rod 42.

The first and second mounting members 42c, 42d are generally only structurally relied upon during installation of the internal fixation rod 42 to hold the respective first and second rod ends 42a, 42b to a pedicle screw 50 (FIGS. 12A-12B) before a surgeon fixes the respective first and second rod ends 42a, 42b.

Preferably, the fixation rod 42 deviates medially and dorsally between the first rod end 42a and the second rod end 42b. The fixation rod 42 deviates medially and dorsally to ease installation or adjustment of material or devices through the foramen 104 between adjacent vertebrae 100. Preferably, the deviation of the fixation rod 42 is generally arcuate, and a dorsal side of the fixation rod 42 is generally convex.

The internal fixation rod 22, 32, 42 can be formed of a rigid material such as a metal, composite or polymeric material. The internal fixation rod 22, 32, 42 can also be formed of a flexible or resilient material such as a flexible metal, a flexible metal composite, a flexible carbon-fiber composite or a flexible or resilient polymeric material. The internal fixation rod 22, 32, 42 can also be formed of combinations thereof.

Optionally, a central portion of the elongate body 23, 33, 43 of the fixation rod 22, 32, 42 may be substituted with a flexible material, a resilient material or any other structure allowing motion such as a spring, a cord, a dynamic stabilization device, an artificial facet or the like, without departing from the invention.

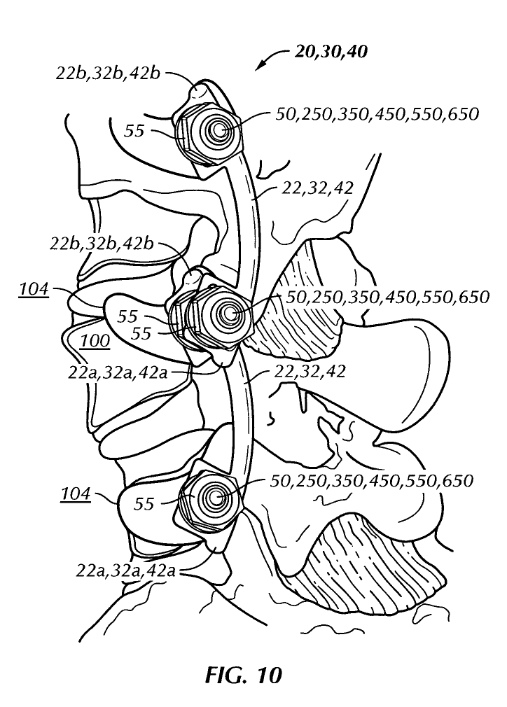

FIGS. 12A-12B show a first pedicle screw 50 for use with the preferred embodiments of the present invention. The first pedicle screw 50 has a bone-mating thread 50b at a first end 50c, a first mating thread 50a at a second end 50d and a longitudinal axis L defined between the first end 50c and the second end 50d. Generally, the pedicle screw 50 is one solid piece with the bone thread or bone-mating thread 50b machined, cast or tapped at the distal portion, and a machine or other mating thread 50a machined, cast or tapped at a proximal portion. But, the pedicle screw 50 may include multiple pieces that are assembled or welded together. The first mating thread 50a is configured to receive a locking nut 55 (FIGS. 11A-11B). The length of the pedicle screw 50 varies depending on the size and shape of the patient, but typically, the pedicle screw is about 5-8 cm in overall length. Likewise, the mating thread portion 50a varies in length depending on how many vertebrae 100 are being fused (i.e., permitting stacking as shown in FIG. 10) and/or if there is need to correct alignment of adjacent vertebrae 100 (e.g., anterolistheis or retrolisthesis). For example, the degree of ventral-dorsal displacement of adjacent vertebrae 100 and/or misalignment of adjacent vertebrae 100 with respect to one another may require a longer mating thread portion 50a. The pedicle screw 50 includes a domed or spherical intermediate-portion 52 which functions both as a mechanical stop when screwing the pedicle screw 50 into a patient's vertebra 100 and as a mounting base for receiving the ends 22a, 22b of the fixation rod 22 (see FIG. 9 for example). The expanded diameter at, for example, domed portion 54 allows the rod 22, 32, 42 to tilt as necessary during installation. The tilting is to minimize or eliminate metal-metal, material-material, metal-bone, material-bone interface stress that would otherwise be caused during the fastening/securing. Optionally, the intermediate shaft 52 includes a smooth portion 53 of about the same or slightly larger diameter as the first mating thread 50a.

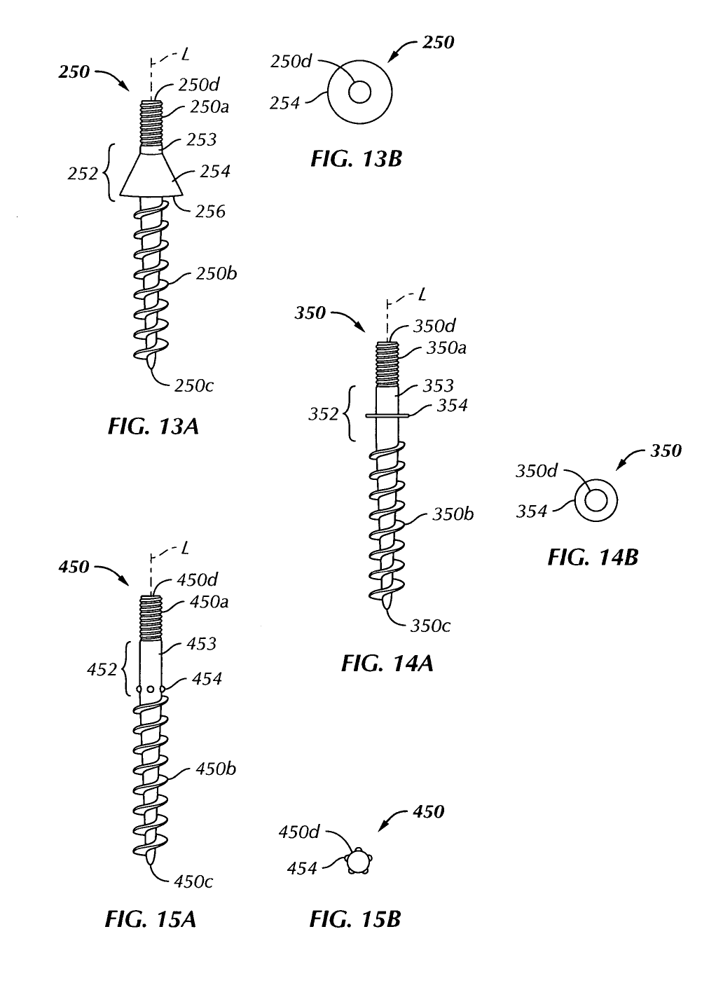

FIGS. 13A-13B show a second pedicle screw 250 for use with the preferred embodiments of the present invention. The second pedicle screw 250 is similar in size, shape and material of construction as the first pedicle screw 50. The second pedicle screw 250 has a bone-mating thread 250b at a first end 250c, a first mating thread 250a at a second end 250d and a longitudinal axis L defined between the first end 250c and the second end 250d. The pedicle screw 250 has an intermediate shaft 252 disposed between the bone-mating thread 250b and the first mating thread 250a. The intermediate shaft 252 includes an upper surface 254 that is generally sloped downwardly and outwardly from the first mating thread and a lower surface 256 that extends generally radially outwardly from the longitudinal axis L of the pedicle screw 250. The expanded diameter at, for example, sloped upper surface 254 allows the rod 22, 32, 42 to tilt as necessary during installation, and the generally flat lower surface 256 provides a stopping function during installation with less chance of bone fracture than a rounded lower surface such as the intermediate section 52 of the first pedicle screw 50. The tilting is to minimize or eliminate metal-metal, material-material, metal-bone, material-bone interface stress that would otherwise be caused during the fastening/securing. Optionally, the intermediate shaft 252 includes a smooth portion 253 of about the same or slightly larger diameter as the first mating thread 250a.

FIGS. 14A-14B depict a third pedicle screw 350 for use with the preferred embodiments of the present invention. The third pedicle screw 350 is similar in size, shape and material of construction as the first pedicle screw 50. The third pedicle screw 350 has a bone-mating thread 350b at a first end 350c, a first mating thread 350a at a second end 350d and a longitudinal axis L defined between the first end 350c and the second end 350d. The third pedicle screw 350 has an intermediate shaft 352 disposed between the bone-mating thread 350b and the first mating thread 350a. The intermediate shaft 352 includes an upper surface 354 that extends generally radially outwardly from the longitudinal axis L of the pedicle screw 350 and a lower surface 356 that extends generally radially outwardly from the longitudinal axis L of the pedicle screw 350. The generally flat lower surface 356 provides a stopping function during installation with less chance of bone fracture than a rounded lower surface such as the intermediate section 52 of the first pedicle screw 50. Optionally, the upper surface 354 may be slightly chamfered or rounded to allow the rod 22, 32, 42 to tilt as necessary during installation. Optionally, the intermediate shaft 352 includes a smooth portion 353 of about the same or slightly larger diameter as the first mating thread 350a.

FIGS. 15A-15B show a fourth pedicle screw 450 for use with the preferred embodiments of the present invention. The fourth pedicle screw 450 is similar in size, shape and material of construction as the first pedicle screw 50. The fourth pedicle screw 450 has a bone-mating thread 450b at a first end 450c, a first mating thread 450a at a second end 450d and a longitudinal axis L defined between the first end 450c and the second end 450d. The fourth pedicle screw 450 has an intermediate shaft 452 disposed between the bone-mating thread 450b and the first mating thread 450a. The intermediate shaft 452 includes a plurality of protuberances 454 disposed around a circumference of the intermediate shaft 452. The protuberances 454 may be hemi-spherical, sloped, partially rounded or the like. The expanded diameter at, for example, the protuberances 454 allows the rod 22, 32, 42 to tilt as necessary during installation. The tilting is to minimize or eliminate metal-metal, material-material, metal-bone, material-bone interface stress that would otherwise be caused during the fastening/securing. Optionally, the intermediate shaft 452 includes a smooth portion 453 of about the same or slightly larger diameter as the first mating thread 450a.

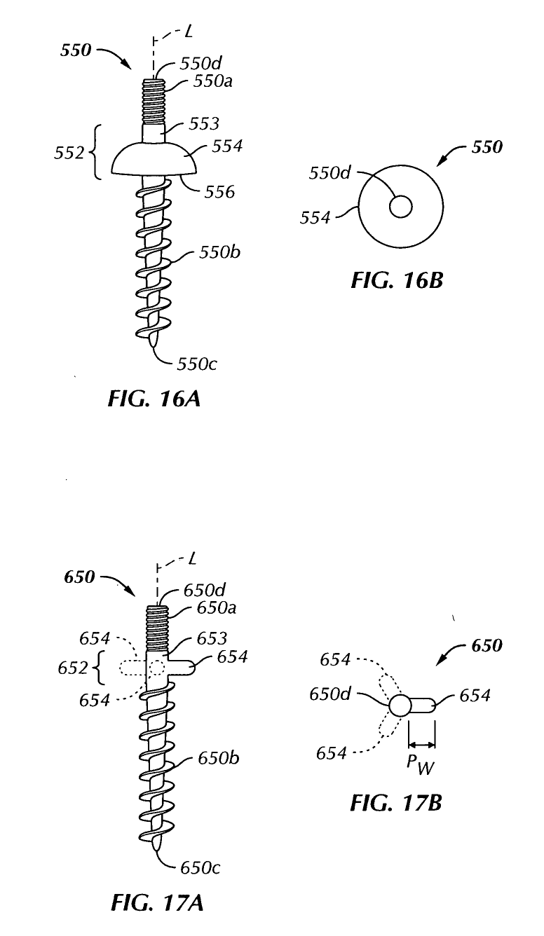

FIGS. 16A-16B depict a fifth pedicle screw 550 for use with the preferred embodiments of the present invention. The fifth pedicle screw 550 is similar in size, shape and material of construction as the first pedicle screw 50. The fifth pedicle screw 550 has a bone-mating thread 550b at a first end 550c, a first mating thread 550a at a second end 550d and a longitudinal axis L defined between the first end 550c and the second end 550d. The fifth pedicle screw 550 has an intermediate shaft 552 disposed between the bone-mating thread 550b and the first mating thread 550a. The intermediate shaft 552 includes an upper surface 554 that extends generally radially outwardly from the longitudinal axis L of the pedicle screw 550 and a lower surface 556 that extends generally radially outwardly from the longitudinal axis L of each pedicle screw 550. The expanded diameter at, for example, rounded hemi-spherical like surface 554 allows the rod 22, 32, 42 to tilt as necessary during installation, and the generally flat lower surface 556 provides a stopping function during installation with less chance of bone fracture than a rounded lower surface such as the intermediate section 52 of the first pedicle screw 50. The tilting is to minimize or eliminate metal-metal, material-material, metal-bone, material-bone interface stress that would otherwise be caused during the fastening/securing. Optionally, the intermediate shaft 552 includes a smooth portion 553 of about the same or slightly larger diameter as the first mating thread 550a.

FIGS. 17A-17B show a sixth pedicle screw 650 for use with the preferred embodiments of the present invention. The sixth pedicle screw 650 is similar in size, shape and material of construction as the first pedicle screw 50. The sixth pedicle screw 650 has a bone-mating thread 650b at a first end 650c, a first mating thread 650a at a second end 650d and a longitudinal axis L defined between the first end 650c and the second end 650d. The sixth pedicle screw 650 has an intermediate shaft 652 disposed between the bone-mating thread 650b and the first mating thread 650a. The intermediate shaft 652 includes a protuberance 654. The protuberance 654 has a width P_{W} that is at least half the diameter or cross-sectional dimension R_{D1}, R_{D2}, R_{D3} of the fixation rod 22, 32, 42. The protuberance 654 may be hemi-spherical, sloped, partially rounded or the like. The expanded diameter at, for example, the protuberance 654 allows the rod 22, 32, 42 to tilt as necessary during installation. The tilting is to minimize or eliminate metal-metal, material-material, metal-bone, material-bone interface stress that would otherwise be caused during the fastening/securing. Optionally, there may be two to four protuberances 654 (phantom in FIG. 17B). By using one to four protuberances 654, the field of view beyond the protuberances 654 is improved over a solid circumferential structure such as intermediate portion 54. Optionally, the intermediate shaft 652 includes a smooth portion 653 of about the same or slightly larger diameter as the first mating thread 650a.

Optionally, each of the pedicle screws 50, 250, 350, 450, 550, 650 may include a grip portion such as a hexagonal or dihexagonal grip for tightening using a nut driver (not shown) or wrench (not shown). Optionally, each of the pedicle screws 50, 250, 350, 450, 550, 650 may include a slot(s) (not shown) at the proximal end 50d, 250d, 350d, 450d, 550d, 650d for installing using a screw driver (not shown), hex-wrench (not shown) Torx-wrench (not shown) or the like. Torx is a Registered Trademark of Camcar Div. of Textron Inc., Providence, Road Island.

Preferably, the pedicle screws 50, 250, 350, 450, 550, 650 are formed of a biocompatible material such as stainless steel, titanium, nickel plated metal, any biocompatible metal or alloy, a biocompatible ceramic, a biocompatible polymeric material or the like.

FIGS. 11A-11B show a locking nut 55 for use with the preferred embodiments of the present invention. The locking nut has a grip portion 55a such as a hexagonal or dihexagonal grip for tightening using a nut driver or wrench. The locking nut 55 further includes a shaped upper surface 55c and a stem portion 55b which protects the first mating threads 50a of a first pedicle screw 50 when a second or additional fixation rod 22 is mounted on top of the locking nut 55. The shaped upper surface 55c of locking nut 55 preferably has a shape similar to the intermediate shaft 52, 252, 352, 452, 552, 652 of the respective pedicle screw 50, 250, 350, 450, 550, 650 to provide a similar installation surface for the next level during multi-level/multi-rod installations (see e.g., FIG. 10). Two adjacent vertebrae 100 would require a single level of fixation (see e.g., FIG. 9), while three adjacent vertebrae 100 would require two levels of fixation with a common screw 50, 250, 350, 450, 550, 650 shared between two-levels (see e.g., FIG. 10) and so on. Such a shaped locking nut 55 allows for one or more additional fixation rods 22 to be mounted on the mating thread portion 50a of the same pedicle screw 50.

FIGS. 4-6 are views of a first internal fixation clamp 58. The first internal fixation clamp 58 is placed over the mating thread portion 50a, 250a, 350a, 450a, 550a, 650a of the pedicle screw 50, 250, 350, 450, 550, 650 after the fixation rod ends 22a, 32a, 42a, 22b, 32b, 42b are placed over the mating thread portion 50a, 250a, 350a, 450a, 550a, 650a. The first internal fixation clamp 58 has two angled projections 58a on the side which will face the rod 22, 32, 42 during installation to function as a clamp forcing the fixation rod 22, 32, 42 against the dome 54 of the first pedicle screw 50; the upper surface 254, 354, 554 of the second, third or fourth pedicle screws 250, 350, 550; against the protuberances 454, 654 of the fourth or sixth pedicle screws 450, 650; or against the shaped upper surface 55c of a locking nut 55 (in a multi-rod installation). The top of the clamp 58 has a dome shape with an ovoid or slotted opening 58b for receiving the mating thread portion 50a, 250a, 350a, 450a, 550a, 650a of the pedicle screw 50, 250, 350, 450, 550, 650. The length of the slotted opening 58b permits the internal fixation rod 22, 32, 42 to tilt on the intermediate shaft 52, 252, 352, 452, 552, 652 of the pedicle screw 50, 250, 350, 450, 550, 650 during installation in order to accommodate variable angles for differing configurations and body types.

Optionally, the first internal fixation clamp 58 may simply be a washer, a lock washer or a washer with a protuberance on its lower surface. Additional mounting hardware may also be utilized with or without the first internal fixation clamp 58 such as washers, lock washers or the like.

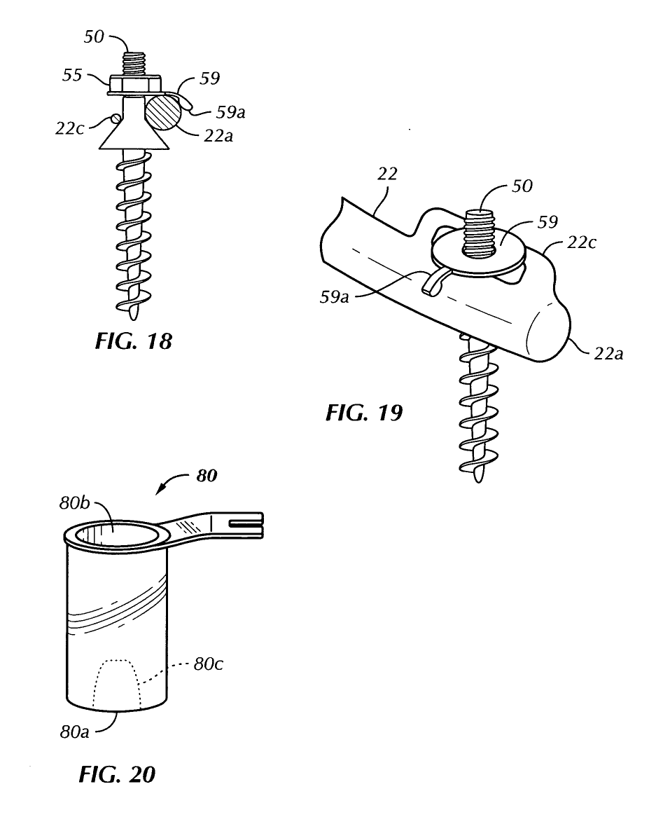

FIGS. 18-19 show a second internal fixation clamp 59. The second internal fixation clamp 59 is placed over the mating thread portion 50a, 250a, 350a, 450a, 550a, 650a of the pedicle screw 50, 250, 350, 450, 550, 650 after the fixation rod ends 22a, 32a, 42a, 22b, 32b, 42b are placed over the mating thread portion 50a, 250a, 350a, 450a, 550a, 650a. The second internal fixation clamp 59 has an angled projection 59a on the side which will face the rod 22, 32, 42 during installation to function as a clamp forcing the fixation rod 22, 32, 42 against the dome 54 of the first pedicle screw 50; the upper surface 254, 354, 554 of the second, third or fourth pedicle screws 250, 350, 550; against the protuberances 454, 654 of the fourth or sixth pedicle screws 450, 650; or against the shaped upper surface 55c of a locking nut 55 (in a multi-rod installation). The top of the second internal fixation clamp 59 has a relatively flat shape with an opening 59b for receiving the mating thread portion 50a, 250a, 350a, 450a, 550a, 650a of the pedicle screw 50, 250, 350, 450, 550, 650. The single projection 59a permits the internal fixation rod 22, 32, 42 to tilt on the intermediate shaft 52, 252, 352, 452, 552, 652 of the pedicle screw 50, 250, 350, 450, 550, 650 during installation in order to accommodate variable angles for differing configurations and body types.

Optionally, the second internal fixation clamp 59 may simply be a washer, a lock washer or a washer with a protuberance on its lower surface. Additional mounting hardware may also be utilized with or without the second internal fixation clamp 59 such as washers, lock washers or the like.

The open ended U-shape or Y-shape of the rods 32, 42 or the closed-loop shape of rod 22 may be applied to any small plate or rod to ease installation on a pedicle screw 50, 250, 350, 450, 550, 650 without departing from the present invention.

FIG. 9 shows an internal fixation system 20, 30, 40 in accordance with the various preferred embodiments the present invention. The an internal fixation system 20, 30, 40 includes an internal fixation rod 22, 32, 42 mounted to adjacent vertebrae 100 of a spine 120 using pedicle screws 50, 250, 350, 450, 550, 650, fixation clamps 58, 59 and locking nuts 55. FIG. 10 shows the internal fixation system 20, 30, 40 in accordance with the various preferred embodiments the present invention with a plurality of internal fixation rods 22, 32, 42 mounted to a plurality of adjacent vertebrae 100 of a spine 120.

The internal fixation systems 20, 30, 40 are mounted completely within the human body, and therefore, all of the various components of the internal fixation systems 20, 30, 40 are formed of or coated with a biologically compatible material such as stainless steel, titanium, nickel plated metal, any biocompatible metal or alloy, a biocompatible ceramic, a biocompatible polymeric material or the like.

The fixation system 20, 30, 40 is preferably used in outpatient spine surgery. For example, a surgeon makes an incision between about 10 mm and about 100 mm in span in a posterior region of a patient proximate a first vertebra 100 and a second vertebra 100 of a spine 120 of the patient. The incision is preferably off-center with respect to the posterior-side of the spine 120 of the patient and proximate to the foraminae 104 of the first and second vertebrae 100. The surgeon inserts a distal end 80a of the working tube or channel 80 (FIG. 20) proximate the first vertebra 100 and the second vertebra 100 of the spine 120 accessible through the incision. The working channel 80 permits the surgeon to access the first vertebra 100 and the second vertebra 100 of the spine 120 from a proximal end 80b of the working channel 80. The surgeon then mounts the internal fixation system 20, 30, 40 for securing adjacent vertebrae 100 that includes the rod 22, 32, 42.

Mounting of the internal fixation system 20, 30, 40 is performed by installing pedicle screws 50, 250, 350, 450, 550, 650 into each of the adjacent vertebrae 100. The surgeon may pre-drill the bone of the vertebrae 100. Optionally, but less preferably, the bone-mating thread 50b, 250b, 350b, 450b, 550b, 650b of the pedicle screws 50, 250, 350, 450, 550, 650 may be configured to be self-tapping. A first end 22a, 32a, 42a of the rod 22, 32, 42 is mounted to the mating thread 50a, 250a, 350a, 450a, 550a, 650a of one of the pedicle screws 50, 250, 350, 450, 550, 650, and a second end 22b, 32b, 42b of the rod 22, 32, 42 is mounted to the mating thread 50a, 250a, 350a, 450a, 550a, 650a of the other pedicle screw 50, 250, 350, 450, 550, 650. The internal fixation rod 22, 32, 42 takes a particular tilt to accommodate the particular installation. An internal fixation clamp 58, 59 is mounted over the mating thread 50a, 250a, 350a, 450a, 550a, 650a of each pedicle screw 50, 250, 350, 450, 550, 650 to thereby secure each of the first and second rod ends 22a, 32a, 42a, 22b, 32b, 42b, respectively. A locking nut 55 is mounted to the mating thread 50a, 250a, 350a, 450a, 550a, 650a of each pedicle screw 50 over each internal fixation clamp 58, 59. Then the surgeon fixes the respective first and second rod ends 22a, 32a, 42a, 22b, 32b, 42b by tightening each locking nut 55 as necessary. Additional fixation rods 22, 32, 42, fixation clamps 58, 59 and locking nuts 55 may be repeatedly stacked and mounted, as necessary, on top of the mating thread 50a, 250a, 350a, 450a, 550a, 650a of each pedicle screw 50, 250, 350, 450, 550, 650 for joining a plurality of adjacent vertebrae 100 (see FIG. 10).

Preferably, the procedure is performed with working channels or tubes 80 that include a slot 80c or slots 80c at the distal-most portion of the working channels or tubes 80 for facilitating the complex dexterous work to be performed such as screwing in pedicle screws 50, 250, 350, 450, 550, 650, attaching rods 22, 32, 42, tightening mounting hardware such as nuts 55 or the like. The slots 80c permit sliding longer components such as the pedicle screws 50, 250, 350, 450, 550, 650 and rods 22, 32, 42 into the area of interest, and the slots 80a permit the surgeon to slide the working channel or tube 80 past the pedicle screws 50, 250, 350, 450, 550, 650 without lifting up the working channel or tube 80 to perform installation of the rods 22, 32, 42, clamps 58 and/or nuts 55.

While described herein as being used with a pedicle screw 50, 250, 350, 450, 550, 650, the internal fixation system 20, 30, 40 may also be used with a rod, stud, bolt or other similar mounting hardware. The bone of the vertebrae 100 may be drilled and tapped or drilled and filled with a biocompatible epoxy, acrylic or other biocompatible material that can cure and harden as an alternate to a threaded screw in order to retain a mounting rod, stud, bolt or the like.

From the foregoing, it can be seen that the present invention is directed to an internal fixation system for spine surgery and a method for using the same. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. An internal fixation rod for spine surgery comprising:
an elongate body;
a first rod end;
a second rod end;
a first mounting member disposed proximate the first rod end; and
a second mounting member disposed proximate the second rod end, the first mounting member and the second mounting member being smaller in cross-section than the elongate body.

2. The fixation rod of claim 1, wherein the fixation rod deviates medially and dorsally between the first rod end and the second rod end.

3. The fixation rod of claim 2, wherein the deviation of the fixation rod is generally arcuate and a dorsal side of the fixation rod is generally convex.

4. The fixation rod of claim 1, wherein the fixation rod is formed of at least one of a rigid material, a flexible material and a resilient material.

5. An internal fixation system for spinal surgery comprising:
two pedicle screws, each of the pedicle screws having a bone-mating thread at a first end, a first mating thread at a second end and a longitudinal axis defined between the first end and the second end;
two locking nuts, each of the locking nuts having a second mating thread configured to mate with the first mating thread of each of the pedicle screws; and
a fixation rod having an elongate body, a first rod end, a second rod end, a first mounting member disposed proximate the first rod end and a second mounting member disposed proximate the second rod end, the first mounting member and the second mounting member being smaller in cross-section than the elongate body, the first rod end and the first mounting member being mounted over one of the pedicle screws and secured by one of the locking nuts and the second rod end and the second mounting member being mounted over the other one of the pedicle screws and secured by the other one of the locking nuts.

6. The internal fixation system of claim 5, wherein each of the pedicle screws has an intermediate shaft disposed between the bone-mating thread and the first mating thread.

7. The internal fixation system of claim 6, where the intermediate shaft includes a protuberance.

8. The internal fixation system of claim 7, where the protuberance has a width that is at least half the cross-sectional dimension of the fixation rod.

9. The internal fixation system of claim 6, wherein the intermediate shaft includes an upper surface that is generally sloped downwardly and outwardly from the first mating thread and a lower surface that extends generally radially outwardly from the longitudinal axis of each pedicle screw.

10. The internal fixation system of claim 6, wherein the intermediate shaft includes an upper surface that extends generally radially outwardly from the longitudinal axis of each pedicle screw and a lower surface that extends generally radially outwardly from the longitudinal axis of each pedicle screw.

11. The internal fixation system of claim 6, wherein the intermediate shaft includes a plurality of protuberances disposed around a circumference of the intermediate shaft.

12. The internal fixation system of claim 6, wherein the intermediate shaft includes an upper surface that is generally hemispherical and a lower surface that extends generally radially outwardly from the longitudinal axis of each pedicle screw.

13. The internal fixation system of claim 5, further comprising:
two fixation clamps, each fixation clamp having a slotted opening that receives the mating threaded portion of one of the pedicle screws, the fixation clamp being mounted between each of the locking nuts and the fixation rod.

14. The internal fixation system of claim 13, wherein the fixation clamps each have angled projections that face the fixation rod in order to secure the fixation rod against the pedicle screw.

15. The internal fixation system of claim 5, wherein the fixation rod deviates medially and dorsally between the first rod end and the second rod end.

16. The fixation rod of claim 5, wherein the fixation rod is formed of at least one of a rigid material, a flexible material and a resilient material.

17. A method of securing adjacent vertebrae, the method comprising:
a) accessing a first vertebra and a second vertebra of a spine; and
b) mounting an internal fixation rod to the first vertebra and the second vertebra, the internal fixation rod having an elongate body, a first rod end, a second rod end, a first mounting member disposed proximate the first rod end and a second mounting member disposed proximate the second rod end, the first mounting member and the second mounting member being smaller in cross-section than the elongate body.

18. A method of installing an internal fixation system for securing adjacent vertebrae, the method comprising:
a) making an incision between about 10 mm and about 100 mm in span in a posterior region of a patient proximate a first vertebra and a second vertebra of a spine of the patient;
b) inserting a distal end of a working channel adjacent the first vertebra and the second vertebra of the spine accessible through the incision;
c) accessing the first vertebra and the second vertebra of the spine through the working channel; and
d) mounting the internal fixation system to the first vertebra and the second vertebra, the internal fixation system including a fixation rod having an elongate body, a first rod end, a second rod end, a first mounting member disposed proximate the first rod end and a second mounting member disposed proximate the second rod end, the first mounting member and the second mounting member being smaller in cross-section than the elongate body.

19. The method of claim 18, further comprising:
d-1) installing pedicle screws into each of the adjacent vertebrae; and
d-2) mounting a first end of the rod to one of the pedicle screws and mounting a second end of the fixation rod to the other pedicle screw.

20. The method of claim 19, further comprising:
d-3) mounting a fixation clamp over each pedicle screw to thereby secure each of the first and second rod ends; and
d-4) mounting a locking nut over each fixation clamp.

21. The method of claim 18, wherein the fixation rod deviates medially and dorsally between the first rod end and the second rod end.
